(19) United States
(12) Patent Application Publication
(10) Pub. No.: US 2013/0098369 A1
(43) Pub. Date: Apr. 25, 2013

(54) Treatment pad for treating obstructive sleep apnea syndrome

(71) Applicants: Chen Qiu, Shenzhen (CN); Xin Zhang, Shenzhen (CN)

(72) Inventors: Chen Qiu, Shenzhen (CN); Xin Zhang, Shenzhen (CN)

(21) Appl. No.: 13/317,518

(22) Filed: Oct. 19, 2011

(51) Int. Cl. A61F 5/56

(52) U.S. Cl. 128/845

(57) ABSTRACT

A treatment pad includes a base member, at least one treatment layer, a plurality of neck supporting members, and an adhesive layer. The treatment layer is peripherally extended from the base member, wherein the treatment layer has a mid portion and two side portions. The neck supporting members are longitudinally extended along the side portions of the treatment layer respectively, wherein each of the neck supporting members has a height gradually increasing from the an inner side edge of the corresponding side portion of the treatment layer to an outer side edge of the corresponding side portion of the treatment layer to define an inclined supporting surface of the corresponding neck supporting member. The adhesive layer is provided on the treatment layer and the neck supporting members for attaching the treatment layer and the neck supporting members on a user's neck portion.

### BACKGROUND OF THE PRESENT INVENTION

1. Field of Invention

The present invention relates to a treatment pad, and more particularly to a treatment pad for effectively treating obstructive sleep apnea syndrome so as to mitigate the symptoms thereof

2. Description of Related Arts

Obstructive Sleep Apnea Syndrome (OSAS) is one of the most common types of sleep apnea and is caused by collapse of soft tissues around the upper airway of a human body. This collapse of the soft tissues causes obstruction of the upper airway so that the person suffering from OSAS will experience repetitive pauses in breathing during sleep. These repetitive pauses in breathing during sleep subsequently cause reduction in blood oxygen saturation. Common symptoms include headaches, loss of memory, fatigue, sleep disorder, loss of concentration, and many more. In the long run, persons suffering from OSAS may develop respiratory or coronary diseases, fatty liver stroke, or even heart attack.

Conventional treatment for OSAS include physical intervention in which some sorts of breathing machines are utilized for pumping air through a mask worn over a the patient's mouse, nose or both. This kind of treatment involving wearing a mask during sleep and this inevitably makes the patient uncomfortable during sleep. Moreover, there also disadvantages in association with the use of the breathing machines. The most common disadvantages include noise problems and expensive maintenance costs. As a result of these disadvantages, treatments for OSAS are limited to those having severe symptoms or to the rich. For those having light to medium symptoms, there exists no effective treatment.

### SUMMARY OF THE PRESENT INVENTION

The invention is advantageous in that it provides a treatment pad for effectively treating obstructive sleep apnea syndrome so as to mitigate the symptoms thereof. More specifically, the treatment pad of the present invention provides effective treatment for those having light to medium OSAS symptoms.

Another advantage of the invention is to provide a treatment pad for effectively treating obstructive sleep apnea syndrome, wherein the use of the treatment pad of the present invention does not involve expensive medical equipments or costs.

Another advantage of the invention is to provide a treatment pad for effectively treating obstructive sleep apnea syndrome, wherein the thickness of the treatment pad can be optimally adjusted so as to fit different severity of the patient's OSAS symptoms.

Additional advantages and features of the invention will become apparent from the description which follows, and may be realized by means of the instrumentalities and combinations particular point out in the appended claims.

According to the present invention, the foregoing and other objects and advantages are attained by providing a treatment pad, comprising:
a base member;
at least one treatment layer peripherally extended from the base member, wherein the treatment layer has a mid portion and two side portions;
a plurality of neck supporting members longitudinally extended along the side portions of the treatment layer respectively, wherein each of the neck supporting members has a height gradually increasing from the an inner side edge of the corresponding side portion of the treatment layer to an outer side edge of the corresponding side portion of the treatment layer to define an inclined supporting surface of the corresponding neck supporting member; and
an adhesive layer provided on the treatment layer and the neck supporting members for attaching the treatment layer and the neck supporting members on a user's neck portion.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a sectional side view of a treatment pad according to a preferred embodiment of the present invention.

FIG. 2 is a schematic diagram of the treatment pad according to the above preferred embodiment of the present invention.

FIG. 3 is a schematic diagram of the basic structure of a patient's upper respiratory system.

FIG. 4 is a schematic diagram of the treatment pad according to the above preferred embodiment of the present invention, illustrating that the treatment pad is worn by a patient while sleeping.

### DETAILED DESCRIPTION OF THE PREFERRED EMBODIMENT

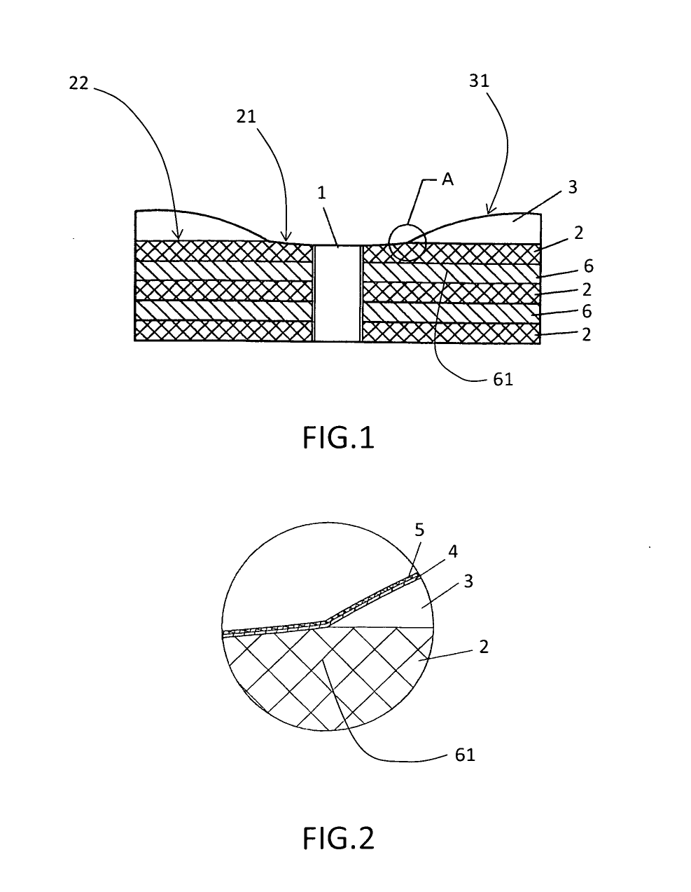

Referring to FIG. 1 to FIG. 4 of the drawings, a treatment pad according to a preferred embodiment of the present invention is illustrated. The treatment pad is for treating obstructive sleep apnea syndrome (OSAS), and comprises a base member 1, a plurality of treatment layers 2, a plurality of neck supporting members 3, and an adhesive layer 4.

Each of the treatment layers 2 is peripherally extended from the base member 1, wherein each of the treatment layers 2 also has a mid portion 21 and two side portions 22.

The plurality of neck supporting members 3 are longitudinally extended along the side portions 22 of the uppermost treatment layer 2 respectively, wherein each of the neck supporting members 3 has a height gradually increasing from the an inner side edge of the corresponding side portion 22 of the uppermost treatment layer 2 to an outer side edge of the corresponding side portion 22 of the uppermost treatment layer 2 to define an inclined supporting surface 31 of the corresponding neck supporting member 3.

The adhesive layer 4 is provided on the uppermost treatment layer 3 and the neck supporting members 3 for attaching the corresponding treatment layer 3 and the neck supporting members 3 on a user's neck portion.

According to the preferred embodiment of the present invention, the treatment pad comprises at least one treatment layer 2 but preferably comprises three treatment layers 2, wherein each of the treatment layers 2 is made of flexible material so that the treatment layers 2 can be easily rolled and unrolled by a user for easily storage and effective protection of the treatment layers 2. Moreover, each of the treatment layers 2 has a through hole formed thereon, wherein the base member 1 is arranged to penetrate the through holes of the treatment layers 2 which are overlappedly stacked so that each of the treatment layers 2 is peripherally extended from the base member 1.

The treatment pad further comprises a plurality of retention layers 6 attached between each two treatment layers 2 for retaining the corresponding treatment layers 2 in a predetermined shape and for reinforcing the treatment layers 2 from being excessively stretched. In other words, each of the retention layers 6 has a predetermined elasticity for normally retaining the treatment layers 2 at a predetermined shape. When the treatment layers 2 are stretched for treatment or other purposes, the retention layers 6 are arranged to exert a retention force for retaining the shape of the treatment layers 2.

Each of the retention layers 6 has a plurality of retention members 61 extended in a longitudinal and a transverse direction of the treatment layer 2 respectively, wherein each of the retention members 61 is also interwoven with a corresponding retention member 61 so as to form a substantially net structure for the corresponding retention layer 6.

As shown in FIG. 1 of the drawings, each of the neck supporting members 3 has a gradual increasing height from the inner side edge of the side portion 22 of the uppermost treatment layer 2 to an outer side edge of the side portion 22 of the uppermost treatment layer 2 to form a butterfly-like appearance of the neck supporting members 3. Moreover, it is worth mentioning that the treatment layers 2 and the retention layers 6 are preferably embodied as having butterfly-like cross sectional shape. However, they may also be embodied as having different cross sectional shapes, such as elliptical cross sectional shape, circular cross sectional shape, rectangular cross sectional shapes, or any other shapes. The attachment between the retention layers 6 and the adjacent treatment layers 2 may be accomplished by a predetermined adhesive agent such as a predetermined amount of glue.

As shown in FIG. 2 of the drawings, the treatment pad further comprises a protective layer 5 detachably attached on the adhesive layer 4 so as to normally protect the adhesive layer 4 from undesirably attracting dirt from ambient environment. The protective layer 5 can be easily detached from the adhesive layer 4 so as to allow the latter to attach to the patient's neck portion for treating OSAS.

In this preferred embodiment, the base member 1 is made of air-permeable rubber material having a thickness of 1 cm to 2 cm (preferably 1 cm). On the other hand, each of the treatment layers 2 is made of polyethylene having a thickness of 0.1 cm to 0.3 cm (preferably 0.2 cm). Each of the neck supporting members 3 is also made of polyethylene having a maximum thickness of 0.4 cm to 0.6 cm (preferably 0.5 cm). Each of the retention layers 6 is made of polyester having a thickness of 0.1 cm to 0.3 cm (preferably 0.2 cm). Finally, the adhesive layer 4 has a thickness of 0.01 cm to 0.02 cm (preferably 0.01 cm).

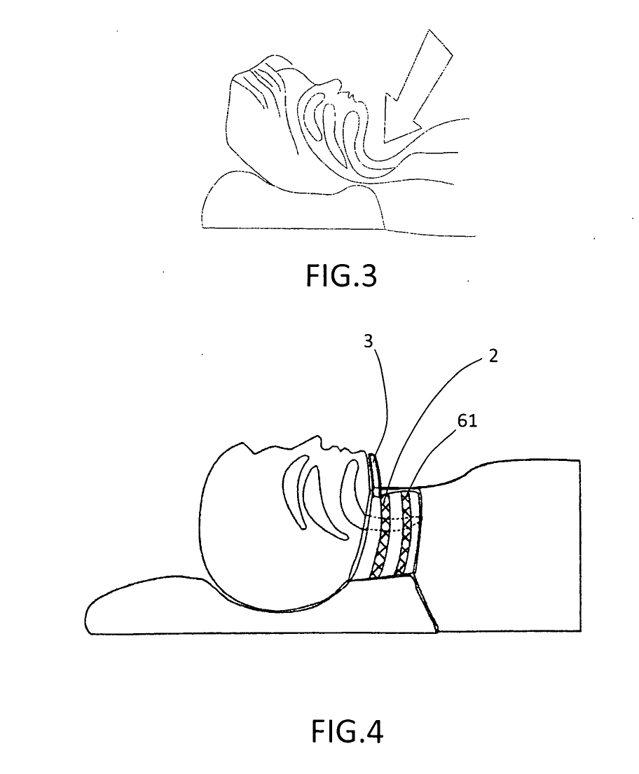

Referring to FIG. 3 of the drawings, when a patient lies down and starts sleeping, the soft tissues in the patient's upper respiratory system may collapse which causes obstruction of the upper airway. The person suffering from OSAS will experience repetitive pauses in breathing during sleep. These repetitive pauses in breathing during sleep subsequently cause reduction in blood oxygen saturation.

Referring to FIG. 4 of the drawings, the operation of the present invention is as follows: when a patient is about to sleep, he or she needs to detach the protective layer 5 from the adhesive layer 4 and attach the uppermost treatment layer 2 and the neck supporting members 3 onto the neck portion of the patient. The neck supporting members 3 are arranged to prevent the soft tissues around the patient's neck portion to collapse downwardly so as to prevent the patient's upper airway from being blocked. Moreover, the treatment layers 2 and the retention layers 6 helps in stretching the soft tissues around the user's neck portion so as to prevent those soft tissues from being collapsed to block the upper airway of the patient.

It is worth mentioning that in this preferred embodiment, three treatment layers 2 have been utilized. However, depending on the severity of the OSAS's symptoms, the number of treatment layers 2 can be altered or adjusted by a medical practitioner or the patient himself so that the thickness of the treatment pad can be adjusted to the optimal.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A treatment pad, comprising:
a base member;
at least one treatment layer peripherally extended from said base member, wherein said treatment layer has a mid portion and two side portions;
a plurality of neck supporting members longitudinally extended along said side portions of said treatment layer respectively, wherein each of said neck supporting members has a height gradually increasing from said an inner side edge of said corresponding side portion of said treatment layer to an outer side edge of said corresponding side portion of said treatment layer to define an inclined supporting surface of said corresponding neck supporting member; and
an adhesive layer provided on said treatment layer and said neck supporting members for attaching said treatment layer and said neck supporting members on a user's neck portion.

2. The treatment pad, as recited in claim 1, further comprising a plurality of treatment layers, wherein each of said treatment layers is made of flexible material so that said treatment layers are capable of being easily rolled and unrolled by a user.

3. The treatment pad, as recited in claim 2, wherein each of said treatment layers has a through hole formed thereon, wherein said base member is arranged to penetrate said through holes of said treatment layers which are overlappedly stacked so that each of said treatment layers is peripherally extended from said base member.

4. The treatment pad, as recited in claim 2, further comprising a plurality of retention layers attached between each two treatment layers for retaining said corresponding treatment layers in a predetermined shape and for reinforcing said treatment layers.

5. The treatment pad, as recited in claim 3, further comprising a plurality of retention layers attached between each two treatment layers for retaining said corresponding treatment layers in a predetermined shape and for reinforcing said treatment layers.

6. The treatment pad, as recited in claim 4, wherein each of the retention layers has a plurality of retention members extended in a longitudinal and a transverse direction of the treatment layer respectively, wherein each of the longitudinally extending retention members is also interwoven with a corresponding transversely extending retention member so as to form a substantially net structure for said corresponding retention layer.

7. The treatment pad, as recited in claim 5, wherein each of the retention layers has a plurality of retention members extended in a longitudinal and a transverse direction of the treatment layer respectively, wherein each of the longitudinally extending retention members is also interwoven with a corresponding transversely extending retention member so as to form a substantially net structure for said corresponding retention layer.

8. The treatment pad, as recited in claim 6, wherein each of said retention layers has a predetermined elasticity for normally retaining said treatment layers at a predetermined shape.

9. The treatment pad, as recited in claim 7, wherein each of said retention layers has a predetermined elasticity for normally retaining said treatment layers at a predetermined shape.

10. The treatment pad, as recited in claim 8, wherein each of said neck supporting members has a gradual increasing height from said inner side edge of said side portion of said uppermost treatment layer to an outer side edge of said side portion of said uppermost treatment layer to form a butterfly-like appearance of said neck supporting members.

11. The treatment pad, as recited in claim 9, wherein each of said neck supporting members has a gradual increasing height from said inner side edge of said side portion of said uppermost treatment layer to an outer side edge of said side portion of said uppermost treatment layer to form a butterfly-like appearance of said neck supporting members.

12. The treatment pad, as recited in claim 1, further comprising a protective layer detachably attached on said adhesive layer so as to normally protect said adhesive layer from undesirably attracting dirt from ambient environment.

13. The treatment pad, as recited in claim 10, further comprising a protective layer detachably attached on said adhesive layer so as to normally protect said adhesive layer from undesirably attracting dirt from ambient environment.

14. The treatment pad, as recited in claim 11, further comprising a protective layer detachably attached on said adhesive layer so as to normally protect said adhesive layer from undesirably attracting dirt from ambient environment.

15. The treatment pad, as recited in claim 14, wherein said base member is made of air-permeable rubber material having a thickness of approximately 1 centimeter to 2 centimeters.

16. The treatment pad, as recited in claim 14, wherein each of said treatment layers is made of polyethylene material having a thickness of approximately 0.1 centimeter to 0.3 centimeter.

17. The treatment pad, as recited in claim 15, wherein each of said treatment layers is made of polyethylene material having a thickness of approximately 0.1 centimeter to 0.3 centimeter.

18. The treatment pad, as recited in claim 17, wherein each of said neck supporting members is made of polyethylene material having a maximum thickness of approximately 0.4 centimeter to approximately 0.6 centimeter.

19. The treatment pad, as recited in claim 18, wherein each of said retention layers is made of polyester material having a thickness of approximately 0.1 centimeter to approximately 0.3 centimeter.

20. The treatment pad, as recited in claim 19, wherein said adhesive layer has a thickness of approximately 0.01 centimeter to 0.02 centimeter.
